(19) United States
(12) Plant Patent Application Publication
(10) Pub. No.: US 2003/0041363 P1
(43) Pub. Date: Feb. 27, 2003

(54) Clematis plant named 'Avalanche'

(76) Inventor: Anthony Robin White, Alresford (GB)

Correspondence Address: MARK P. BOURGEOIS, P.O. BOX 95, OSCEOLA, IN, 46561, US

(21) Appl. No.: 09/939,491

(22) Filed: Aug. 24, 2001

(51) Int. Cl. A01H 5/00

(52) U.S. Cl. PLT/228

(57) ABSTRACT

A new cultivar of Clematis named ‘Avalanche’ that is characterized by bronze-purple stems, shiny green foliage with variable bronze markings, and a profuse display of white flowers in spring. In combination these traits set ‘Avalanche’ apart from all other existing varieties of Clematis known to the inventor

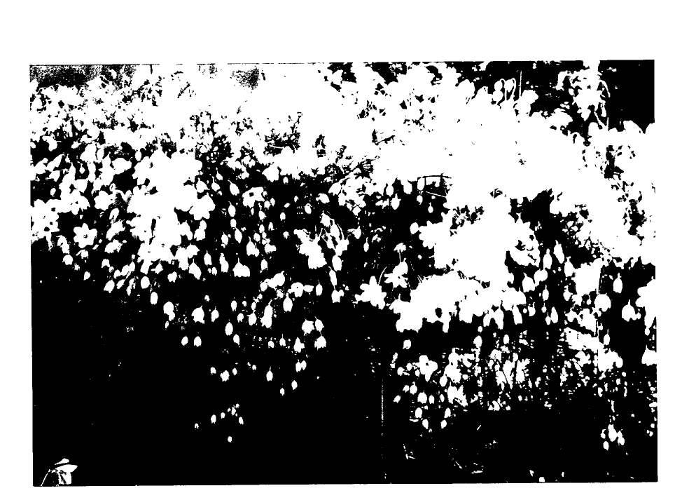

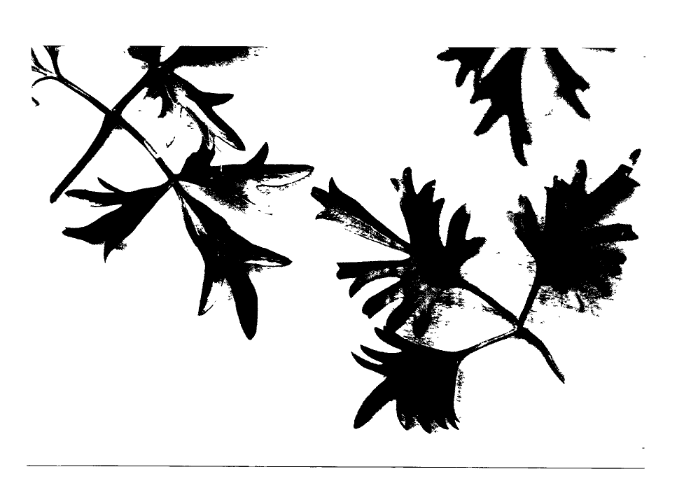

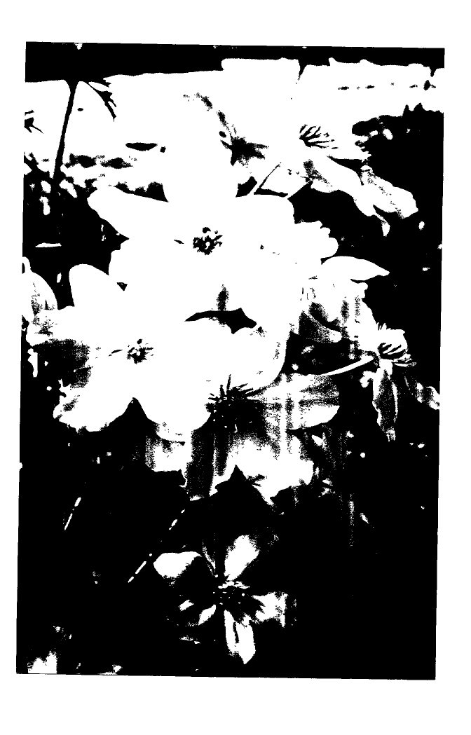

### BACKGROUND OF THE INVENTION

The present invention relates to a new and distinct cultivar of Clematis that is grown for use as an evergreen climber on a frame or wall, or as a ground cover, shrub, tree or container plant. The new cultivar is known botanically as a Clematis xcartmanii and will be referred to hereinafter by the cultivar name ‘Avalanche’.

‘Avalanche’ was bred by the inventor in 1992 by crossing the pollen parent Clematis paniculata with the seed parent Clematis mannoraria. The cross was conducted in a cultivated area of Hampshire, England and ‘Avalanche’ was selected from the resulting seedlings that flowered in 1994. Selection was based on plant vigor and flower. Panicles of cream-white flowers are produced in great profusion on the previous seasons wood. The distinguishing characteristics of ‘Avalanche’ are vigor, leaf shape and large male flowers. Pruning is not required when ‘Avalanche’ is being used as a large shrub, small tree or ground cover for a large area. However, when grown in a container or confined area, pruning is required immediately after flowering. Young foliage may have a temporary light bronze coloration when exposed to low temperatures, that disappears as temperatures rise. However, the color of mature leaves is permanently suffused with bronze markings.

The closest comparison plants are Clematis xcartmanii ‘Spring Sensation’ (unpatented) and Clematis xcartmanii ‘Joe’ (unpatented). ‘Avalanche’ is distinguishable from both comparison plants by its larger flower and increased vigor. In addition to these traits ‘Avalanche’ exhibits foliage that is wider lobed and darker green than the cultivar ‘Joe’.

The first asexual propagation was accomplished in 1994 using cuttings. The first asexual propagation was accomplished by the inventor in Hampshire, England. Since that time, under careful observation, the distinguishing characteristics have been determined stable and uniform in successive generations.

### SUMMARY OF THE INVENTION

The following represent the distinguishing characteristics of the new Clematis cultivar ‘Avalanche’. In combination these traits set ‘Avalanche’ apart from all other existing varieties of Clematis known to the inventor. ‘Avalanche’ has not been tested under all possible conditions and phenotypic differences may be observed with variations in environmental, climatic, and cultural conditions, however, without any variance in genotype.

1. At maturity Clematis ‘Avalanche’ exhibits bronze-purple stems and shiny green broad-lobed foliage with variable dark bronze markings.

2. Clematis ‘Avalanche exhibits increased vigor over any other Clematis xcartmanii variety.

3. In spring Clematis ‘Avalanche’ produces large cream-white flowers on the previous seasons wood.

4. Clematis ‘Avalanche’ is a hardy woody climber that can withstand temperatures of minus 10° Centigrade.

5. Clematis ‘Avalanche’ is dioecious and male.

6. Clematis ‘Avalanche’ is five meters in height at maturity when unpruned.

### BRIEF DESCRIPTION OF THE DRAWINGS

The accompanying color photographs illustrate the distinguishing traits of the new cultivar. The plants in the photographs are twenty months old were grown out of doors in Hampshire, England in three-gallon containers. The photograph on sheet 1 illustrates entire plants in bloom from a side perspective. The photograph on sheet 2 is a close-up view of the foliage. The photograph on sheet 3 is a close-up view of the flowers. All photographs are taken using conventional techniques and although foliage colors may appear different from actual colors due to light reflectance, they are as accurate as possible by conventional photography.

### BOTANICAL DESCRIPTION OF THE PLANT

The following is a detailed description of the Clematis cultivar named ‘Avalanche’. Data was collected in Arroyo Grande, Calif. from the inventor and from plants that were fourteen to twenty months old and grown in three-gallon containers out of doors. Color determinations are in accordance with The Royal Horticultural Society Colour Chart except where general color terms of ordinary dictionary significance are used. The growing requirements are similar to the species.

Botanical classification: Clematis xcartmanii ‘Avalanche’.

Common name: Clematis or vase vine.

Uses: Container plant, climber for frame or wall, ground cover, shrub or small tree for large area.

Parentage: ‘Avalanche’ is a hybrid resulting from the induced hybridization of the following plants:

Female parent.—Clematis mannoraria.

Male parent.—Clematis paniculata.

Type: Suffruticose perennial.

Vigor: Vigorous. Grows approximately 2 meters a year during the first two years.

Habit: Recumbent when used as a ground cover and scandent when given a wall or frame for support.

Height (at maturity): 5 meters in height.

Width (at maturity): 35 cm. in width.

Hardiness: Hardy to minus 10° Centigrade.

Propagation: Cuttings.

Root system: Fibrous.

Soil: Plant in well-drained humus-rich soils with adequate moisture.

Sunlight: Plant in semi-shade to full sun.

Plant sexuality: Dioecious.

Time to initiate roots: 6-8 weeks are required to produce roots on an initial cutting.

Crop time: Approximately 14 months are required to produce a finished three-gallon container from a rooted cutting.

Seasonal interest: Cream-white flowers in spring.

Stem:

Branching arrangement.—Divaricate.

Branching habit.—Recumbent on the ground and scandent with support.

Internode length.—10-13 cm. between nodes.

Stem diameter.—0.50 cm. in diameter.

Primary stem length.—120 cm. long.

Secondary stem length.—14 cm. in length.

Shape.—Cylindrical and fluted.

Surface.—Strigose.

Primary stem color.—200A.

Secondary stem color.—183A.

Trunk dimensions.—5 cm. in height from surface of soil and 1 cm. in width.

Trunk color.—200A.

Foliage:

Type.—Evergreen.

Leaf arrangement.—Opposite.

Leaf division.—Bitemate.

Leaf shape.—Trifoliate and slightly depressed along mid-vein creating a V-shape in cross-section.

Leaf base.—Rounded.

Leaf apex.—Apiculate and mucro at tips of mature lobes.

Leaf venation.—Pinnate with prominent mid-vein.

Vein color (adaxial surface).—137A.

Vein color (abaxial surface).—137A.

Leaf surface (adaxial).—Glabrous and shiny.

Leaf surface (abaxial).—Glabrous and matte.

Leaf attachment.—Petiolate.

Petiole dimensions.—6-10 mm. in length and 1.5 mm. in diameter.

Petiole color.—166A.

Petiole shape.—Tortuous when plant is used as a climber.

Leaf margin.—Entire.

Leaf texture.—Thick and leather-like.

Leaf length.—3-4 cm. in length.

Leaf width.—2-3 cm. in width.

Young leaf color (adaxial surface).—136B,C.

Young leaf color (abaxial surface).—137B.

Young leaf color (margin and apex).—183A.

Mature leaf color (adaxial surface).—166A suffused over 136B,C.

Mature leaf color (abaxial surface).—166A suffused over 136B,C.

Foliar fragrance.—Absent.

Flower:

Inflorescence.—Panicle.

Flower form.—Solitary.

Color of peduncle.—144A.

Peduncle length.—8.5 cm. in length.

Peduncle diameter.—0.20 cm. in diameter.

Self cleaning or persistent.—Self cleaning.

Dimensions of flower.—7.50 cm. in length and 7.50 cm. in width and 1 cm. in depth.

Flower shape.—Discoid.

Flower sexuality.—Male.

Aspect.—Facing upward.

Petals.—Apetalous.

Tepals.—Six in number.

Color of tepals.—155C.

Tepal shape.—Obovate.

Tepal surface.—Undulate.

Tepal margin.—Entire with a notched apex and lateral margins involute.

Tepal length.—3.5 cm. in length.

Tepal width.—2 cm.in width.

Unfused or fused.—Tepals are unfused.

Blooming period.—April to May.

Bud color.—145B.

Bud shape.—Oval.

Bud dimensions.—2 cm. in length and 1 cm. in width.

Quantity of flowers.—Approximately 11 per stem.

Fragrance.—Faintly sweet.

Reproduction organs:

Stamens.—Sixty or more.

Arrangement of stamens.—Spirally arranged.

Color of stamens.—145A.

Dimensions of stamen.—9 mm. in length and 1.5-2 mm in diameter.

Dimensions of anther.—1 mm. in width and 1 mm. in length.

Pollen color.—157A.

Anther.—2 mm. in length and 1.5 mm. in width.

Anther color.—157A.

Anther shape.—Flattened.

Seed: Seed production has not been observed.

1. A new and distinct variety of Clematis plant named ‘Avalanche’ as described and illustrated.
